(19) United States
(12) Patent Application Publication
(10) Pub. No.: US 2012/0239122 A1
(43) Pub. Date: Sep. 20, 2012

(54) APPARATUS AND METHOD FOR HOT-COLD CONTRAST THERAPY

(76) Inventors: Rong Dong, Alameda, CA (US); Mary Hui Dong, Alameda, CA (US)

(21) Appl. No.: 13/423,067

(22) Filed: Mar. 16, 2012

Related U.S. Application Data
(60) Provisional application No. 61/465,329, filed on Mar. 17, 2011.

(51) Int. Cl. A61F 7/08; A61F 7/10

(52) U.S. Cl. 607/100; 607/114; 607/104

(57) ABSTRACT

The present invention teaches an apparatus and method for physical therapy using prompt changing temperature from hot to cold. Its application in proper conditions may promote human body's self recovery function, restore balance for soft tissues and relieve inner pressure.

### REFERENCE TO RELATED APPLICATION

The present application claims priority to the provisional Appl. Ser. No. 61/465,329 filed on Mar. 17, 2011, the entire content of which is hereby incorporated by reference.

### FIELD OF THE INVENTION

The present invention relates generally to medical devices and methods, and more particularly to an integrated optical and electrical system and associated method for treating muscle or tissue disorders and other symptoms using prompt hot-cold contrast application.

### BACKGROUND OF THE INVENTION AND THE RELATED ARTS

1. Heat Therapy

Heat therapy is the application of heat above a certain temperature to the body for pain relief and health. Traditionally it takes the form of a hot cloth, hot water, heated rock, ultrasound, heating pad, hydrocollator pack, whirlpool bath, cordless FIR heat therapy wrap, and many various others. It is beneficial to those with arthritis and stiff muscles and injuries to the muscles and deep tissues of the skin. Heat is also an effective self-care treatment for conditions such as rheumatoid arthritis.

It has been well realized that heat therapy is useful for muscle spasms, myalgia, fibromyalgia, contracture and bursitis. Heat therapy is commonly used for rehabilitation purposes. The therapeutic effects of the controlled heat include increasing the extensibility of collagen tissues; decreasing joint stiffness; reducing pain; relieving muscle spasms; reducing inflammation, edema, and aids in the post acute phase of healing; and increasing blood flow. The increased blood flow to the affected area provides proteins, nutrients, and oxygen for better healing.

Moist heat is one application of heat therapy. In this type of practice, heat is typically applied by placing very warm, wet towels on the relevant body part. It is usually more effective at warming tissues than dry heat because water transfers heat more quickly than air. This results in the perception that the tissue is heated more deeply, which increases the effect on muscles, joints, and soft tissues.

The newest breed of heat therapy device combines a carbon fiber heater with a cordless rechargeable battery and is built into the specific body wrap such as shoulder wrap or back wrap for target heat therapy. It has been used as an alternative to chemical or plugged-in heating pads that are also used for menstrual cramping relief.

Infrared radiation is another type of heat application for therapy. Its advantage over direct contact is that radiation can heat the target area directly where the blood capillaries and neuron terminals exist. When heat comes from a direct contact source it has to heat the external layer of the skin, and heat is transferred to the deeper layer by conduction. Heat conduction needs a temperature gradient to proceed, and there is a maximum temperature that can be safely used, usually around 42° C. Infrared (IR for short), however, can provide heat in a more effective way. IR is the part of the electromagnetic radiation specter comprised between 0.78 μm and 1 mm wavelength. It is usually divided into three sections; IR-A, from 0.78 to 1.4 μm; IR-B, from 1.4 to 3 μm; and IR-C, from 3 μm to 1 mm. IR radiation is more useful than the visible radiation for heating human body because human body absorbs most of it, compared to a strong reflect on of visible light. IR's penetration depth in human skin is dependent of wavelength. IR-A is the most penetrating, and reaches some millimeters, IR-B penetrates into the dermis (about 1 mm), and IR-C is mostly absorbed in the external layer of the epidermis (estratum corneum). For this reason the lamps used for therapeutic purposes produce mainly IR-A radiation.

Heat creates higher tissue temperatures, which produces vasodilatation that increases the supply of oxygen, and nutrients and the elimination of carbon dioxide and metabolic waste. Because heat is a vasodilator, it should be avoided in tissues with inadequate vascular supply, in case of acute injury, in bleeding disorders, in tissues with a severe lack of sensitivity, and in scars.

Another use is the treatment of infection and cancers by the use of heat. Cancer cells and many bacteria have poor mechanisms for adapting to and resisting the physiological stresses of heat, and are more vulnerable to heat-induced death than normal cells.

2. Cold Therapy

Cold therapy, also know as cold compression therapy combines two of the principles of rest, ice, compression and elevation (RICE) to reduce pain and swelling from a sports or activity injury to soft tissues and recommended by orthopedic surgeons following surgery. It is especially useful for sprains, strains, pulled muscles and pulled ligaments.

Cold compression is a combination of cryotherapy and static compression, commonly used for the treatment of pain and inflammation after acute injury or surgical procedures.

Cryotherapy, the use of ice or cold in a therapeutic setting, has become one of the most common treatments in orthopedic medicine. The primary reason for using cryotherapy in acute injury management is to lower the temperature of the injured tissue, which reduces the tissue's metabolic rate and helps the tissue to survive the period following the injury. It is well documented that metabolic rate decreases by application of cryotherapy.

Static compression is often used in conjunction with cryotherapy for the care of acute injuries. To date, the primary reason for using compression is to increase external pressure on the tissue to prevent edema formation or swelling. This occurs by hindering fluid loss from the vessels in the injured area, making it more difficult for fluids to accumulate. Ice with compression is significantly colder than ice alone due to improved skin contact and increased tissue density caused by extended static compression. Tissue reaches its lowest temperature faster and the tissue maintains the cool even after treatment ends.

There is a continuous cold therapy device, also called ice machine, available on the market. The device circulates ice water through a pad. However, its uses have raised safety concerns because excessive cooling/icing time and lack of temperature control may cause skin and tissue damages. Reported injuries range from frost-bite to severe tissue damage resulting in amputation.

Studies have shown that the body activates the hunting response after only 10 minutes of cryotherapy, at temperatures less than 49° F. The hunting response is a cycle of vasoconstriction (decreased blood flow), then vasodilatation (increased blood flow) that increases the delivery of oxygen and nutrient rich blood to the tissue. Increased blood flow can slow cell death, limit tissue damage and aid in the removal of cellular debris and waste products. Under normal circumstances the hunting response would be essential to tissue health out only serves to increase pain, inflammation and cell death as excess blood is forced into the area.

Cold compression wraps using either refreezable ice or gel are a much safer product as such products do not exceed the cooling/icing time recommended by the established medical community. Many of the ice wraps available use adjustable elastic straps to aid in compression over the injured areas. Most ice wraps have a built-in protective layer so ice is not applied directly to the skin which can result in a burn to the area sometimes known as a “cryoburn”.

3. Contrast Bath Therapy

Contrast bath therapy, also known as “hot/cold immersion therapy”, is a form of treatment where a limb or the entire body is immersed in ice water followed by the immediate immersion of the limb or body in warm water. The theory behind contrast bath therapy is that the warm water causes vasodilatation of the blood flow in the limb or body followed by the cold water which causes vasoconstriction. The effect is to pump out inflammation. Contrast bathing can be used to reduce swelling around injuries or to aid recovery from exercise.

There is no existing art which provides a device for prompt hot-cold contrast therapy using optical and electrical means.

### SUMMARY OF THE INVENTION

The present invention discloses and teaches an apparatus for providing prompt hot-cold contrast therapy on a patient. The apparatus includes a thermotherapy unit which applies heat to surrounding areas of a targeted area of the patient and then gradually increases temperature in the targeted area, a cryotherapy unit which applies compressed cold media, a controlling unit which includes one or more sensors to catch the patient's natural response to a maximum tolerance of heat application from the thermotherapy unit, a first switch for controlling the thermotherapy unit's heat supply, a second switch for controlling the cryotherapy unit's release of cold media. As soon as the temperature in the targeted area approaches to the patient's maximum tolerance, the controlling unit turns off the thermotherapy unit's heat application to the target area and turns on the cryotherapy unit's release of cold media. In one preferred embodiment, the thermotherapy unit includes a parabolic dish which provides heat to the target area. The parabolic dish is coupled to the apparatus' body through an adjustable conduit. The parabolic dish includes a heat source which locates at the parabolic dish's focal point. Optionally, the heat source is an array of electrical bulbs or LEDs which provides IR or visible light. The parabolic dish has a number of outlets which are connected to a container containing cold media, the container being included in the apparatus's body. The cold media's release from the outlets is controlled by the controlling unit. The container for containing cold media can be replaceable. The one or more sensors are fixed on the parabolic dish and face the target area. The cold media is any of: cold air, cold liquid and cold gel.

### BRIEF DESCRIPTION OF THE DRAWINGS

FIG. 1 is schematic block diagram illustrating the basic components of the apparatus according to the present invention;

FIG. 2A is a schematic diagram illustrating a therapy head 20 and its application to a target area 30 of a human body; and

FIG. 2B is a schematic diagram illustrating an exemplary structure of the therapy head of FIG. 2A.

### DESCRIPTION OF THE INVENTION

While the present invention may be embodied in many different forms, designs or configurations, for the purpose of promoting an understanding of the principles of the invention, reference will be made to the embodiments illustrated in the drawings and specific language will be used to describe the same. It will nevertheless be understood that no limitation or restriction of the scope of the invention is thereby intended. Any alterations and further implementations of the principles of the invention as described herein are contemplated as would normally occur to one skilled in the art to which the invention relates.

The present invention provides an apparatus and method for prompt hot-cold contrast therapy using optical and electrical means. By combining the beneficial effects of heat and cold therapy techniques, it heals the injured areas effectively.

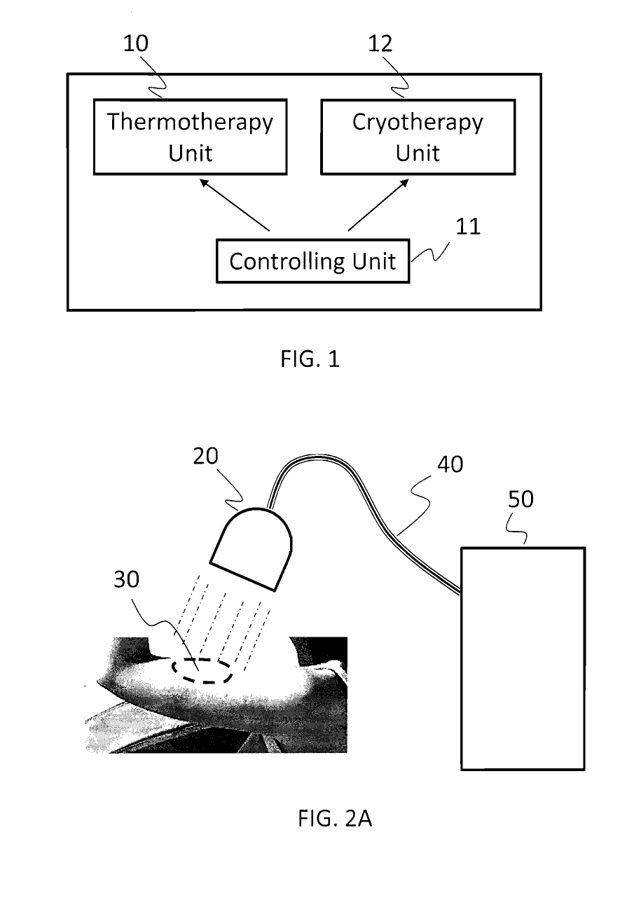

FIG. 1 is schematic block diagram illustrating the basic components of the apparatus according to the present invention. The apparatus includes (1) a thermotherapy unit 10, (2) a controlling unit 11, and (3) a cryotherapy unit 12.

The thermotherapy unit 10 provides heat therapy. It first applies medium heat to the surrounding areas of a target area of a human body and then gradually increases the temperature in the target area. The application of superficial heat to human body can achieve positive effects similar to traction. Heat therapy promotes relaxation via lengthening of the collagen tissues within the structures such as muscle nuts, ligaments and tendons, and thereby inducing a stretching process. It can also improve the flexibility of tendons and ligaments, reduce muscle spasms, alleviate pain, elevate blood flow and boost metabolism.

The controlling unit 11 includes (1) one or more sensors to catch the patient's natural spontaneous response to the maximum tolerance of heat application from the thermotherapy unit 10, and (2) at least two electrical/mechanical switches, one for controlling the heating source of the thermotherapy unit 10 and the other for controlling the release of cold media from the cryotherapy unit 11. For vigorous heat application to a given site, the location of the peak temperature applied will be coincide with the site such that maximally tolerated tissue temperatures will be obtained there without burning elsewhere. The controlling unit 11 monitors the temperature through one or more sensors. When the temperature approaches to the patient's maximum tolerance, the patient's spontaneous response is usually a sudden movement of the part under the heat treatment. As soon as the sensors detect the movement, the thermotherapy unit 10 is turned off and at the same time, the release of cold media by the cryotherapy unit 12 is initiated.

The cryotherapy unit 12 provides cold therapy by applying compressed cold air or cold liquid or other media such as gel. Once the temperature at the target areas reaches the patient's maximum tolerated limit, which means that the traction process is completed, the heat application from the thermotherapy unit 10 is quickly shut off, and cold media from the cryotherapy unit 12 is immediately applied to the target area. Cold therapy produces vasoconstriction, which slows blood circulation, reducing inflammation, muscle spasm, and pain. More importantly, the prompt hold-cold comparison initiates the human body's self-organizing potentials and has the muscles and nerves reorganized to a normal condition.

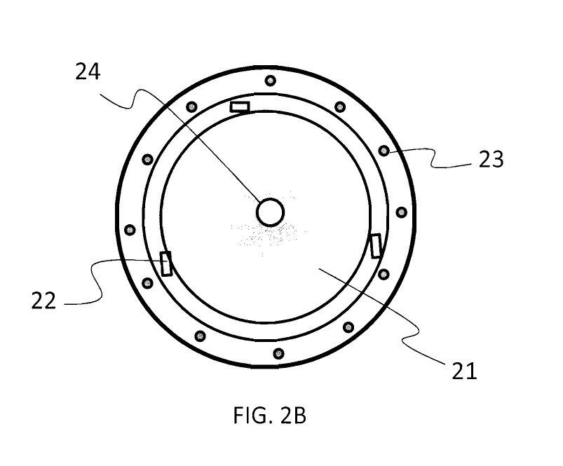

FIG. 2A is a schematic diagram illustrating a therapy head 20 and its application to a target area 30 of a human arm. The therapy head 20 is connected to the device body 50 through an adjustable arm 40. The adjustable arm 40 includes electrical wires and a conduit for conveying cold media.

FIG. 2B is a schematic diagram illustrating an exemplary structure of the therapy head 20. The therapy head 20 includes a parabolic dish 21 which provides heat, a number of sensors 22 which fixed on the dish and facing the target area, and a number of outlets 23 which provide cold media. The sensors 22 are electronically connected to one or processors in the controlling unit 11.

In a typical embodiment, the parabolic dish 21 has a heat source 24 which locates at its focal point. The heat source 24 can be an array of electrical bulbs or LEDs which provides IR or visible light.

In a typical embodiment, the cold media is contained in a replaceable container installed in the device body 50. The release or dispensation of cold media is electronically controlled by the controlling unit 11.

In application, the user adjusts the therapy head 20 to the proper position facing a target area 30, and turns on the device. The therapy head 20 provides heat to the target area. The temperature at the target area increases. When the patient gives a natural spontaneous response to the maximum tolerance temperature, the sensors 22 notifies the controlling unit 11 which turns off the thermotherapy unit 10 promptly and at the same time turns on the cryotherapy unit 12. A sufficient amount of cold media is quickly released or dispensed from the outlets 23 to the target area 30. Thus, a treatment session, which includes a heating phase and cooling phase, is completed. In practice, the time difference from the heat application is turned off to the application of cold media to the skin is less than 1 second.

The other aspect of the present invention is a method to provide hold-cold therapy. The method comprises the steps of: (1) providing a means for heating a target area of a patient's body; (2) providing a means to detect the patient's spontaneous response to the patient's maximal tolerance to the heat; (3) providing a means to apply cold media to the target area as soon as the patient's spontaneous response to the patient's maximal tolerance to the heat is detected.

While one or more embodiments of the present invention have been illustrated above, the skilled artisan will appreciate that modifications and adoptions to those embodiments may be made without departing from the scope and spirit of the present invention.

1. An apparatus for providing prompt hot-cold contrast therapy on a patient, comprising:
a thermotherapy unit which applies heat to surrounding areas of a targeted area of the patient and then gradually increases temperature in the targeted area;
a cryotherapy unit which applies compressed cold media;
a controlling unit which comprises one or more sensors to catch the patient's natural response to a maximum tolerance of heat application from the thermotherapy unit, a first switch for controlling the thermotherapy unit's heat supply, and a second switch for controlling the cryotherapy unit's release of cold media;
wherein when the temperature in the targeted area approaches to the patient's maximum tolerance, the controlling unit turns off the thermotherapy unit's heat application to the target area and turns on the cryotherapy unit's release of cold media.

2. The apparatus of claim 1, wherein said thermotherapy unit comprises a parabolic dish which provides heat to the target area, said parabolic dish being coupled to the apparatus' body through an adjustable conduit.

3. The apparatus of claim 2, wherein said parabolic dish comprises a heat source which locates at said parabolic dish's focal point.

4. The apparatus of claim 3 wherein said heat source is an array of electrical bulbs or LEDs which provides IR or visible light.

5. The apparatus of claim 2, wherein said parabolic dish comprises a number of outlets which are connected to a container containing cold media, said container being included in the apparatus's body, and wherein said cold media's release from the outlets is controlled by said controlling unit.

6. The apparatus of claim 5 wherein said container is a replaceable container.

7. The apparatus of claim 2, wherein said one or more sensors are fixed on the parabolic dish and face the target area.

8. The apparatus of claim 1, wherein said cold media is any of cold air, cold liquid and cold gel.

9. A method for providing prompt hot-cold contrast therapy on a patient, comprising steps of:
providing a thermotherapy unit which applies heat to surrounding areas of a targeted area of the patient and then gradually increasing temperature in the targeted area;
providing a cryotherapy unit which applies compressed cold media; and
providing a controlling unit which comprises one or more sensors to catch the patient's natural response to a maximum tolerance of heat application from the thermotherapy unit, a first switch for controlling the thermotherapy unit's heat supply, and a second switch for controlling the cryotherapy unit's release of cold media;
wherein when the temperature in the targeted area approaches to the patient's maximum tolerance, the controlling unit turns off the thermotherapy unit's heat application to the target area and turns on the cryotherapy unit's release of cold media.

10. The method of claim 9, wherein said thermotherapy unit comprises a parabolic dish which provides heat to the target area.

11. The method of claim 10, wherein said parabolic dish comprises a heat source which locates at said parabolic dish's focal point.

12. The method of claim 11, wherein said heat source is an array of electrical bulbs or LEDs which provides IR or visible light.

13. The method of claim 10, wherein said parabolic dish comprises a number of outlets which are connected to a container containing cold media, and wherein said cold media's release from the outlets is controlled by said controlling unit.

14. The method of claim 13, wherein said container is a replaceable container.

15. The method of claim 2, wherein said one or more sensors are fixed on the parabolic dish and face the target area.

16. The method of claim 1, wherein said cold media is any of: cold air, cold liquid and cold gel.

17. A method for providing prompt hot-cold contrast therapy on a patient, comprising steps of:
using a heating device to gradually apply heat to a target area of a patient's body;
detecting the patient's spontaneous response to the patient's maximum tolerance of heat application; and
concurrently shutting off heat application and dispensing cold media to the target area immediately the patient's spontaneous response is detected;
wherein said cold media is any of: cold air, cold liquid and cold gel.

18. The method of claim 17, wherein heating device is a parabolic dish with a heat source at its focal point, said heat source being an array of electrical bulbs or LEDs which provides IR or visible light.

19. The method of claim 18, wherein said parabolic dish comprises a number of outlets for dispending cold media.

20. The method of claim 19, wherein said parabolic dish comprises one or more sensors for detecting the patient's spontaneous response.
